(19) United States
(12) Patent Application Publication
(10) Pub. No.: US 2014/0348748 A1
(43) Pub. Date: Nov. 27, 2014

(54) BETA-AMYLOID IMAGING AGENTS, METHODS OF MANUFACTURE, AND METHODS OF USE THEREOF

(71) Applicants: Lisheng Cai, Derwood, MD (US); Victor W. Pike, Bethesda, MD (US)

(72) Inventors: Lisheng Cai, Derwood, MD (US); Victor W. Pike, Bethesda, MD (US)

(21) Appl. No.: 14/345,004

(22) PCT Filed: Sep. 13, 2012
(86) PCT No.: PCT/US2012/055124

§ 371 Date: Apr. 23, 2014

Related U.S. Application Data
(60) Provisional application No. 61/535,569, filed on Sep. 16, 2011.

(51) Int. Cl. A61K 51/04; G01N 33/68; C07D 417/04

(52) U.S. Cl. CPC A61K 51/0455; C07D 417/04; G01N 33/6896; G01N 2333/4709
USPC 424/1.89; 546/270.1; 424/1.85; 424/1.81; 436/87

(57) ABSTRACT

Derivatives of benzothiazolylbenzeneamines useful as imaging agents for nuclear imaging such as positron emission tomography of beta-amyloids are described. Methods of detecting amyloid using the compounds are described. Also disclosed is a radiolabeling method for selected compounds.

### FIELD OF THE DISCLOSURE

The present disclosure relates to amyloid binding compounds, specifically compounds that are useful as radioligands for the detection of Aβ amyloid aggregates in Alzheimer's Disease (AD) patients, particularly by PET imaging.

### BACKGROUND

Alzheimer's Disease (AD) is the most common cause of dementia and is characterized by progressive impairment in cognitive function and behavior. This progressive, irreversible brain disorder affects millions of lives and imposes a devastating burden on the health care around the world. Over the past two decades, significant progress has been made in deciphering the pathogenesis of AD and developing new therapeutic approaches. The pathological features of AD include neuritic plaques composed of amyloid β peptides (Aβ), neurofibrillary tangles of hyper-phosphorylated tau, and neurotransmitter deficits. Recent efforts of managing AD have been focused on the prevention of production, aggregation, and deposition of Aβ in the brain and the acceleration of clearance of Aβ from the brain.

Non-invasive detection and quantitation of amyloid deposits in the brain has been used to develop anti-amyloid therapies. Direct imaging of amyloid load in vivo in patients with AD is useful for the early diagnosis of AD and the development and assessment of treatment strategies. To this end, compounds suitable for in vivo imaging of amyloid deposits in human brains have been developed. Among these compounds are monoclonal antibodies against Aβ and Aβ peptide fragments, but these compounds have had limited uptake by the brain when tested in patients with AD. Putrescine-gadolinium-Aβ has been injected into transgenic mice over-expressing amyloid, and this has resulted in labeling observed with MRI. Amyloid deposition can also be non-invasively imaged and quantitated with a radiotracer that readily enters the brain and selectively binds to amyloid deposits.

The small molecule approach for amyloid imaging has so far been the most successful. Some of the promising compounds used to image amyloid are based on Congo red, thioflavin, and stilbene, and compounds such as [¹⁸F]1-(6-((2-fluoroethyl)-methyl)amino)naphthalen-2-yl)ethylidene)malononitrile ([¹⁸F]FDDNP). Amyloid-β (Aβ) imaging with N-methyl-¹¹C-2-(4′-methylamino-phenyl)-6-hydroxy-benzothiazole (¹¹C-6-OH-BTA-1; also known as ¹¹C-PIB) has also been used. The binding of different derivatives of Congo red and thioflavin has been studied in human autopsy brain tissue and in transgenic mice. Two compounds in advanced testing are fluorine-18-labelled Amyvid™ (florbetapir) from Eli Lilly ((E)-4-(2-(6-(2-(2-(2-[¹⁸F]fluoroethoxy)ethoxy)ethoxy)pyridin-3-yl)vinyl)-N-methylaniline, and flutemetamol from GE (2-(3-fluoro-4-(methylamino)phenyl)benzo[d]thiazol-6-ol).

Two tertiary amines, [¹⁸F]FEM-IMPY [N-(2-fluoroethyl)-4-(6-iodo-H-imidazo[1,2-a]pyridin-2-yl)-N-methylbenzeneamine], and its 3-fluoropropyl analog, [¹⁸F]FPM-IMPY, have been previously evaluated as β-amyloid radioligands, as reported by Cai, L., et al., “Synthesis and evaluation of two ¹⁸F-labeled 6-iodo-2-(4′-N,N-dimethylamino)phenylimidazo[1,2-a]pyridine derivatives as prospective radioligands for beta-amyloid in Alzheimer's disease,” in J. Med. Chem., Volume 47, pp. 2208-2218 (2004). After intravenous injection of either radioligand into rodent or monkey, there is rapid and high uptake of radioactivity into brain with an SUV (standardized uptake value, % I.D. kg/g) of about 160 followed by biphasic clearance with a fast and a very slow component. The radioligands were rapidly metabolized by processes involving de-alkylation of the tertiary aromatic amino group, culminating in defluoridation and high uptake of radioactivity in bone. Tetra-deuteration of the fluoroethyl group did not lead to a significant reduction in the residual brain radioactivity, but reduced the bone uptake of radioactivity, presumably due to an isotope effect on metabolism.

The effective management of Alzheimer's disease requires tools to diagnose, monitor, treat, and prevent the disease. There accordingly remains a need for amyloid imaging agents with high specificity of binding to beta amyloid, low background noise, better entry into the brain, and improved labeling efficiencies.

### SUMMARY

In one aspect, a compound of Formula 1, or a pharmaceutically acceptable salt thereof, is disclosed:

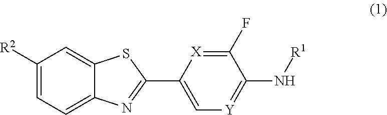

wherein

X is CH or N;

Y is CH or N;

wherein X and Y are not the same;

R¹ is H, C₁-C₆ alkyl, C₁-C₆ haloalkyl, C₁-C₆ hydroxyalkyl, C₇-C₁₃ arylalkylene, carbamoyl C₁-C₆ alkyl, or Y and R¹ are joined to form a C₂-C₄ alkylene, C₂-C₄ alkenylene, or thio-C₁-C₃ alkylene linkage between Y and the amine nitrogen to which R¹ is attached; and

R² is H, OH, halo, C₁-C₆ alkyl, C₁-C₆ haloalkyl, C₁-C₆ alkoxy, C₁-C₆ haloalkoxy, C₁-C₆ alkylthio, or C₆-C₁₂ aryl.

In another aspect, included is a pharmaceutical composition comprising a compound of Formula 1 and a pharmaceutically acceptable carrier.

In a further aspect, a method of detecting amyloid deposits in a patient comprises administering to the patient a pharmaceutical composition comprising a detectable quantity of a compound of Formula 1, and detecting the compound in the subject.

In a still further aspect, a method of detecting and/or quantifying amyloid in biopsy or post-mortem tissue comprises contacting a preparation of biopsy or post-mortem tissue with a compound of Formula 1, wherein the compound comprises a detectable label, and detecting the compound in the tissue.

The invention is further illustrated by the following Figures, Description, Examples, and Claims.

### BRIEF DESCRIPTION OF THE FIGURES

FIG. 1 shows a synthetic scheme for obtaining an ¹⁸F-radiolabeled compound of Formula 1a, compound 1.6.

FIG. 2 shows a synthetic scheme for obtaining ¹⁸F-radiolabeled compounds of Formula 1, compounds 1.4, 1.5 and 1.6.

FIG. 3 shows the results of in vitro binding assays of AD brain homogenates.

FIGS. 4 to 6 show time-activity curves of PET images of compounds 1.6, 1.5, and 1.4 of Formula 1, respectively, in monkeys.

FIG. 7 shows autoradiographs of compound 1.6 in healthy and AD brain tissue slides

### DETAILED DESCRIPTION

This invention is directed to novel amyloid binding compounds that are useful as radiolabeled imaging agents for amyloid imaging, particularly amyloid imaging in the brain. In particular, the present disclosure provides analogs of benzo[d]thiazole useful as radioligands for the detection of Aβ amyloid aggregates in Alzheimer's Disease patients. The compounds advantageously do not undergo rapid defluoridation and do not produce residual radioactivity in the brain. The compounds are further stable, and readily synthetically available.

In an embodiment, a novel class of amyloid imaging agents is represented by Formula 1:

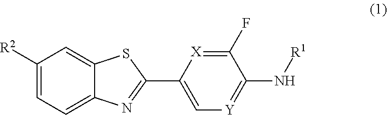

wherein

X is CH or N;

Y is CH or N;

wherein X and Y are not the same;

R¹ is H, C₁-C₆ alkyl, C₁-C₆ haloalkyl, C₁-C₆ hydroxyalkyl, C₇-C₁₃ arylalkylene, carbamoyl C₁-C₆ alkyl, or Y and R¹ are joined to form a C₂-C₄ alkylene, C₂-C₄ alkenylene, or thio-C₁-C₃ alkylene linkage between Y and the amine nitrogen to which R¹ is attached; and

R² is H, OH, halo, C₁-C₆ alkyl, C₁-C₆ haloalkyl, C₁-C₆ alkoxy, C₁-C₆ haloalkoxy, C₁-C₆ alkylthio, or C₆-C₁₂ aryl.

In all of the embodiments herein when R¹ and Y are joined to form a thio-alkylene linkage, Y is CH, and the sulfur atom of the thio-alkylene linkage is bonded to Y.

In an embodiment of the compounds of Formula 1, X is N and Y is CH, or Y is N and X is CH.

Further in an embodiment of the compounds of Formula 1, R¹ is C₁-C₄ alkyl, C₁-C₄ fluoroalkyl, or Y and R¹ are joined to form a C₂-C₃ alkylene, C₂-C₃ alkenylene, or thio-C₁-C₂ alkylene linkage between Y and the amine nitrogen to which R¹ is attached. Specifically, R¹ is H, methyl, ethyl, fluoromethyl, fluoroethyl, or Y and R¹ are joined to form a C₂-C₃ alkylene or thio-C₁-C₂ alkylene linkage between Y and the amine nitrogen to which R¹ is attached.

In another embodiment of the compounds of Formula 1, R² is H, OH, C₁-C₆ alkyl, C₁-C₆ alkoxy, or C₆-C₁₂ aryl, and specifically in some embodiments R² is R² is H, OH, C₁-C₆ alkyl, or C₁-C₆ haloalkoxy. In still other embodiments, R² is H, OH, or C₁-C₄ alkoxy, specifically methyl.

In a specific embodiment of the compounds of Formula 1,

X is N and Y is CH, or Y is N and X is CH;

R¹ is C₁-C₄ alkyl, C₁-C₄ fluoroalkyl, or Y and R¹ are joined to form a C₂-C₃ alkylene, C₂-C₃ alkenylene, or thio-C₁-C₂ alkylene linkage between Y and the amine nitrogen to which R¹ is attached; and

R² is H, OH, C₁-C₆ alkyl, C₁-C₆ alkoxy, or C₆-C₁₂ aryl. Alternatively in this embodiment, R² is H, OH, C₁-C₆ alkyl, C₁-C₆ alkoxy, or C₆-C₁₂ aryl, or R² is H, OH, C₁-C₆ alkoxy, or C₁-C₆ haloalkoxy, or alternatively, R² is H, OCH₃, or OH.

In another specific embodiment of the compounds of Formula 1,

X is N and Y is CH;

R¹ is H, methyl, ethyl, fluoromethyl, fluoroethyl, or Y and R¹ are joined to form a C₂-C₃ alkylene or thio-C₁-C₂ alkylene linkage between Y and the amine nitrogen to which R¹ is attached; and

R² is H, OH, C₁-C₆ alkyl, C₁-C₆ alkoxy, or C₆-C₁₂ aryl. Alternatively in this embodiment, R² is H, OH, C₁-C₆ alkyl, C₁-C₆ alkoxy, or C₆-C₁₂ aryl, or R² is H, OH, C₁-C₆ alkoxy, or C₁-C₆ haloalkoxy, or alternatively R² is H, OCH₃, or OH.

In still another specific embodiment of the compounds of Formula 1,

X is N and Y is CH;

R¹ and Y are joined to form a C₂-C₃ alkylene, C₂-C₃ alkenylene, or thio-C₁-C₂ alkylene linkage between Y and the amine nitrogen to which R¹ is attached; and

R² is H, OH, C₁-C₆ alkyl, C₁-C₆ alkoxy, or C₆-C₁₂ aryl. Alternatively in this embodiment, R² is H, OH, C₁-C₆ alkyl, C₁-C₆ alkoxy, or C₆-C₁₂ aryl, or R² is H, OH, C₁-C₆ alkoxy, or C₁-C₆ haloalkoxy, or alternatively R² is H, OCH₃, or OH.

In another specific embodiment of the compounds of Formula 1, X is N and Y is CH; R¹ is H, methyl, ethyl, fluoromethyl, fluoroethyl, or Y and R¹ are joined to form a C₂-C₃ alkylene or thio-C₁-C₂ alkylene linkage between Y and the amine nitrogen to which R¹ is attached; and R² is H, OH, or C₁-C₄ alkoxy.

In still another specific embodiment of the compounds of Formula 1, X is N and Y is CH; R¹ is H, methyl, or Y and R¹ are joined to form a C₂-C₃ alkylene or thio-C₁-C_{2 alkylene linkage between Y and the amine nitrogen to which R}¹ is attached; and R² is H, OH, or OCH₃.

In a specific embodiment of the compounds of Formula 1, X is N and Y is CH; R¹ and Y are joined to form an ethylene, propylene, thiomethylene, or thioethylene linkage wherein any S atom is adjacent the Y group; and R² is OH, OCH₃ or H.

In a specific embodiment of the compounds of Formula 1, X is N and Y is CH; R¹ is methyl; and R² is OH, OCH₃ or H.

Specific compounds within the scope of Formula 1 are shown in Table 1. In structures 1.7 to 1.18, R¹ and Y are joined. When R¹ contains S, it is adjacent the Y—CH— group.

TABLE 1

| Compound | R¹ | Y | X | R² |
|---|---|---|---|---|
| 1.1 | H | CH | N | H |
| 1.2 | H | CH | N | OCH₃ |
| 1.3 | H | CH | N | OH |
| 1.4 | CH₃ | CH | N | H |
| 1.5 | CH₃ | CH | N | OCH₃ |
| 1.6 | CH₃ | CH | N | OH |
| 1.7 | CH₂CH₂ | CH | N | H |
| 1.8 | CH₂CH₂ | CH | N | OCH₃ |
| 1.9 | CH₂CH₂ | CH | N | OH |
| 1.10 | CH₂CH₂CH₂ | CH | N | H |
| 1.11 | CH₂CH₂CH₂ | CH | N | OCH₃ |
| 1.12 | CH₂CH₂CH₂ | CH | N | OH |
| 1.13 | CH₂S | CH | N | H |
| 1.14 | CH₂S | CH | N | OCH₃ |
| 1.15 | CH₂S | CH | N | OH |
| 1.16 | CH₂CH₂S | CH | N | H |
| 1.17 | CH₂CH₂S | CH | N | OCH₃ |
| 1.18 | CH₂CH₂S | CH | N | OH |

In an embodiment, the amyloid binding compounds of Formula 1 are radiolabeled, for example with ¹⁹F, ¹³C, ¹⁸F, ¹¹C, ⁷⁵Br, ⁷⁶Br, or ¹²³I, specifically ¹⁸F or ¹¹C. Such compounds can be obtained via synthesis with an appropriate radiolabeled starting material, or substitution of a group with a radiolabeled atom in an intermediate or the final product. In an embodiment, the fluorine at the position adjacent to X in the phenyl ring in Formula 1 is ¹⁸F as shown in Formula 1a:

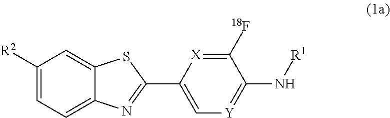

wherein X, Y, R¹, and R² are as defined in Formula 1.

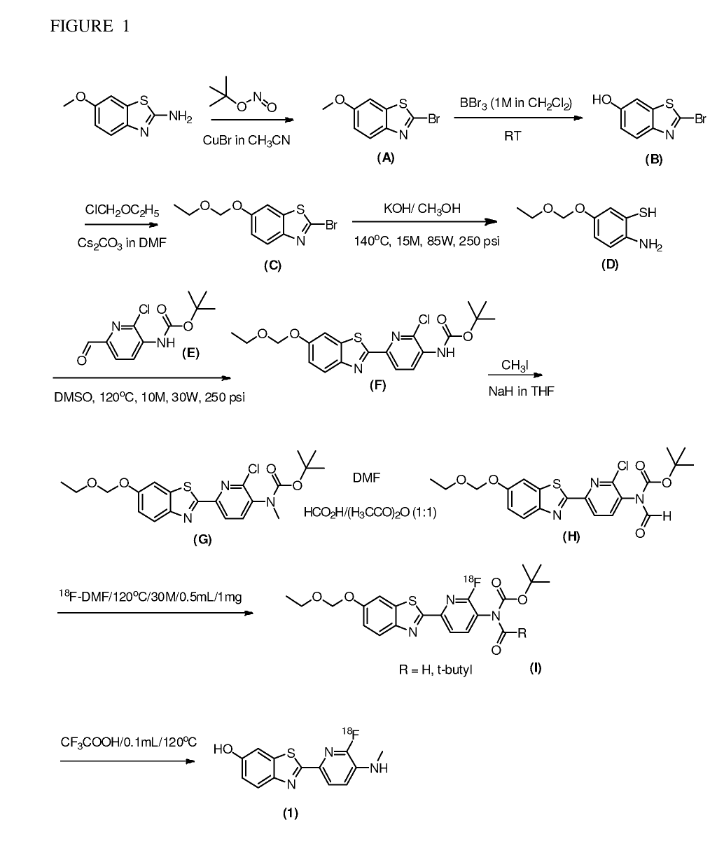

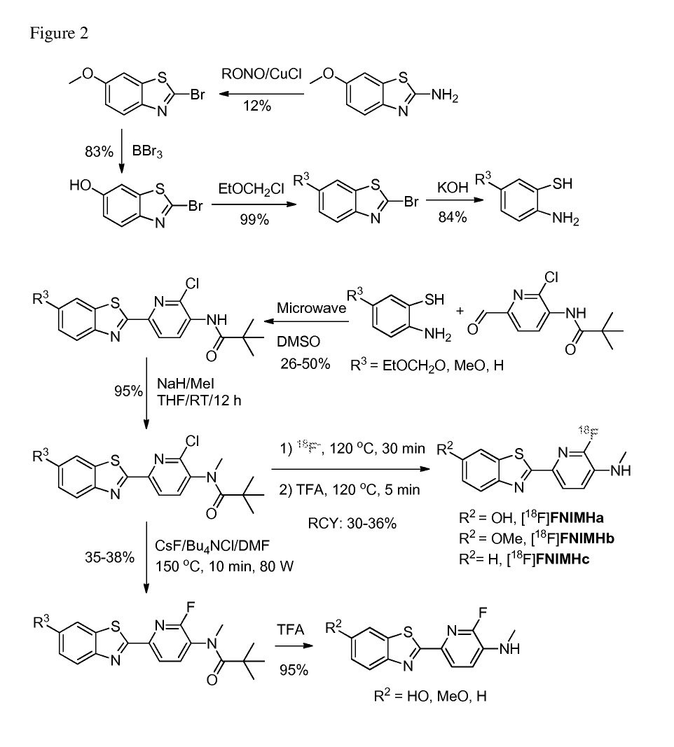

Compounds of Formulas 1 and 1a can be synthesized according to the synthetic scheme shown in FIGS. 1 and 2. The scheme in FIG. 1 produces the compound of Formula 1 wherein X is N, Y is CH, R¹ is methyl, and R² is hydroxyl, compound 1.6. While the synthetic scheme is specific to this compound it can be extended to other compounds of Formula 1 using the guidance herein and the knowledge of one of ordinary skill in the art.

Accordingly, as shown in FIG. 1, a commercially available starting material (6-methoxy-benzothiazole) is brominated by diazotization with tert-butanol nitrite and copper bromide, then demethylated using BBr₃ in dichloromethane to produce the brominated benzothiazole (A). While FIG. 1 illustrates a synthesis of a specific compound (I), the scheme shown in the Figure may be used to produce other compounds (I) using the guidance set forth herein.

Other routes were unsuccessful. For example, demethylation of 6-methoxybenzo[d]thiazol-2-amine using BBr₃ proceeded easily to afford 2-aminobenzo[d]thiazol-6-ol as shown in Scheme A.

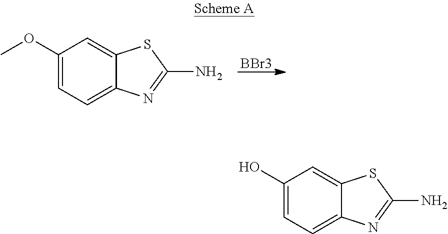

However, the reactivity of the amino group and phenol group is so close that no reagent that was examined (such as ClCH₂OC₂H₅, acetyl chloride, and mixed anhydride of formic acid and acetic anhydride) differentiated the two groups.

When the amino group of 6-methoxybenzo[d]thiazol-2-amine was protected first with acetic anhydride as shown in Scheme B, the problem becomes how to differentiate the phenol group and the amide NH group. When ClCH₂OC₂H₅ was reacted with the amide as shown in Scheme A, both groups reacted either randomly or at the same time.

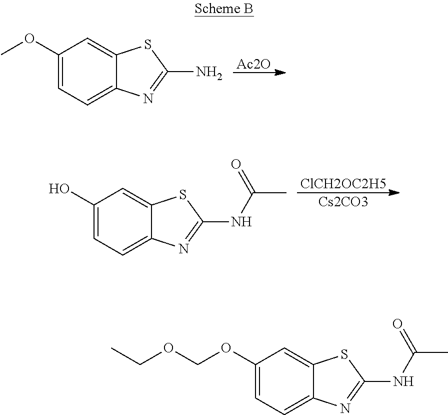

When the desired compound was purified, the thiazole ring resisted hydrolysis under a variety of conditions. This route was discarded.

When a reported procedure was used to synthesize 2-bromo-6-methoxybenzo[d]thiazole as shown in Scheme C, dibrominated product was isolated.

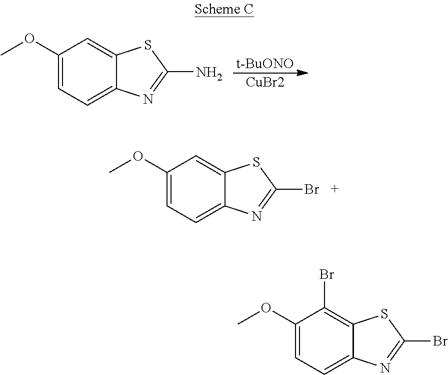

Even when a stoichiometric amount of CuBr₂ was used, a mixture of monobromo- and dibromo-compounds was generated. The two products have identical properties, such as identical spots on TLC, and identical elutions via HPLC with a variety of C18 HPLC columns. The only differentiation that could be seen was through ¹H NMR. In order to form monobrominated product, CuBr was used instead as shown in the Figure. Under anaerobic conditions, no dibromo product was formed.

The phenol group of the bromobenzothiazoles (B) was then protected using ClCH₂OCH₂CH₃ under weakly basic conditions to form the protected bromobenzothiazole (C) prior to forming the 2-aminobenzenethiol (D) by hydrolysis.

Hydrolysis of the protected bromobenzothiazole (C) to form the 2-aminobenzenethiols (D) was effected in KOH/methanol via microwave irradiation. The standard procedure for the synthesis of these derivatives is via prolonged hydrolysis with concentrated KOH aqueous solution. This condition works well for 6-methoxybenzo[d]thiazol-2-amine. However, if the starting material was not soluble in water, no reaction was observed. The reaction was developed using methanol or ethanol as solvent and using microwave heating instead of conventional heating. When 2-bromo-6-methoxybenzo[d]thiazole was used instead of 6-methoxybenzo[d]thiazol-2-amine, the reaction was much faster.

The 2-amino-benzenethiol (D) was then reacted with a Boc-protected aldehyde precursor (E), again under microwave heating, to produce the benzo[d]thiazol-2-yl 3-pyridine derivatives (F). A general procedure using equal amounts of aldehyde and 2-amino-benzenethiol in DMSO was employed to generate the desired products. This is a one-pot, two step reaction, with the condensation of the 2-amino benzenethiol with aldehyde, and oxidation of the intermediate to benzo[d]thiazol-2-yl 3-pyridine. All these compounds are fluorescent with a different coefficient.

The benzo[d]thiazol-2-yl 3-pyridine (F) was subsequently methylated to produce aryl amine (G). The secondary amine was converted to the corresponding amide by reaction with a mixed anhydride. The secondary amine can also be protected with a pivaloyl group. Introducing N—CHO group in the aryl amine (G) to produce the formamide (H) was achieved using mixed anhydride generated from equal volume of formic acid and acetic anhydride. The 3-methylamino group only reacted with mixed anhydride to generate formamide.

Fluoride substitution of the chloride group of the amide (H) to produce the fluoro-substituted benzo[d]thiazol-2-yl 3-pyridine derivatives (I) was achieved through use of excess anhydrous CsF. It was observed that in compounds using the —CH₂OCH₂CH₃ protecting groups as shown in compound (H), no clear difference was observed between the formamide and pivalamide group. This is in contrast with what was observed for radiolabeling reaction using methyl as a phenol protecting group as discussed below.

The methylated product was radiolabeled by reaction with ¹⁸F-dimethyl formamide (DMF) in the presence of trifluoroacetic acid. Hydrolysis of amides can be achieved through both acid and base catalysis. K₃PO₄/CsF did not hydrolyze formamide, but hydrolyzed the pivalamide group. The stronger base hydrolyzed the formamide, but hydrolysis of the 2-fluoro group of the pyridine ring proceeded even faster. Acidic conditions hydrolyze both formamide and pivalamide group under rather mild conditions.

An advantage of the derivatives described herein relative to the prior art compound [¹¹C]PIB (6-OH-BTA-1,2-[4-(methylamino)phenyl]-6-hydroxybenzothiazole) is that the derivatives can be easily labeled with [¹⁸F], which has a half-life of about two hours as compared with twenty minutes for [¹¹C]. The substituent of the amide has profound effect on the yield of radiolabeling reaction. Formyl amide gave low yield on radiolabeling in the first step. Most of the ¹⁸F for formamide was introduced in the second step when acid was added. Under acidic conditions, the pyridine nitrogen is protonated, this activates the 2-chloro group, and ¹⁸F is introduced at this step. However, when pivalamide was used, 30-35% yield of ¹⁸F was introduced in the first step, probably due to the larger size of the pivalamide group preventing the coplanar arrangement with the pyridine ring, making the 2-chloro group much more reactive.

The compounds of Formula 1, particularly Formula 1a, can be used to detect the presence and location of amyloid deposits in an organ or body area, specifically the brain, of a patient. The method comprises administration of a detectable quantity of a pharmaceutical composition containing a compound of Formula 1, specifically Formula 1a, as disclosed herein or a pharmaceutically acceptable salt thereof, to a patient. A “detectable quantity” means that the amount of the compound that is administered is sufficient to enable detection of binding of the compound to amyloid. An “imaging effective quantity” means that the amount of the compound that is administered is sufficient to enable imaging of the compound bound to amyloid.

The compounds of Formula 1, particularly Formula 1a, are used in non-invasive nuclear medicine imaging techniques such as magnetic resonance spectroscopy (MRS) or imaging (MRI), or gamma imaging such as positron emission tomography (PET) or single-photon emission computed tomography (SPECT). Imaging is used to quantify amyloid deposition in vivo. The term “in vivo imaging” refers to a method which permits the detection of a labeled amyloid binding compound as described herein. For nuclear medicine imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of labeled compound along with a large excess of unlabeled, but otherwise chemically identical compound. A “subject” is a mammal, specifically a human, and most specifically a human having or suspected of having dementia.

For purposes of in vivo imaging, the compounds of Formula 1 are labeled. The type of detection is a major factor in selecting a given label. For instance, labeling with ¹¹C and ¹⁸F are particularly suitable for in vivo PET imaging with the compounds of Formula 1. The type of instrument used will guide the selection of the radionuclide or stable isotope. For instance, the radionuclide chosen should have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabeled compounds can be detected using nuclear medicine imaging wherein emitted radiation of the appropriate wavelength is detected.

The labeled amyloid binding compounds are thus useful for in vivo imaging and quantification of amyloid deposition. The amyloid binding compounds may be labeled with ¹⁹F or ¹³C for MRS/MRI, with ¹⁸F, ¹¹C, ⁷⁵Br, or ⁷⁶Br for PET, or with ¹²³I for SPECT by any of several techniques known to the art. Radiolabeling techniques are well known in the art. The compounds also may be radiolabeled with known metal radiolabels, such as Technetium-99m (^{99m}Tc). Modification of the substituents to introduce ligands that bind such metal ions can be effected without undue experimentation by one of ordinary skill in the radiolabeling art. The metal radiolabeled amyloid binding compound can then be used to detect amyloid deposits. Preparing radiolabeled derivatives of ^{99m}Tc is well known in the art.

The method may be used to diagnose AD in mild or clinically confusing cases. This technique would also allow longitudinal studies of amyloid deposition in human populations at high risk for amyloid deposition such as familial AD. A method that allows the temporal sequence of amyloid deposition to be followed can determine if deposition occurs long before dementia begins or if deposition is unrelated to dementia. This method can be used to monitor the effectiveness of therapies targeted at preventing amyloid deposition.

Generally, the dosage of the labeled amyloid binding compounds will vary depending on considerations such as age, condition, sex, and extent of disease in the patient, contraindications, if any, concomitant therapies and other variables, to be adjusted by a physician skilled in the art. Dosage can vary from 0.001 μg/kg to 10 μg/kg, preferably 0.01 μg/kg to 1.0 μg/kg.

Administration to the subject may be local or systemic and accomplished intravenously, intra-arterial, intrathecally (via the spinal fluid) or the like. Administration may also be intradermal or intracavitary, depending upon the body site under examination. After a sufficient time has elapsed for the compound to bind with the amyloid, for example 30 minutes to 48 hours, the area of the subject under investigation is examined by imaging techniques such as MRS/MRI, SPECT, planar scintillation imaging, PET, and emerging imaging techniques. The exact protocol may vary depending upon factors specific to the patient, as noted above, and depending upon the body site under examination, method of administration and type of label used; the determination of specific procedures would be routine to the skilled artisan. In one embodiment, for brain imaging, the amount (total or specific binding) of the bound radioactively amyloid binding compound is measured and compared (as a ratio) with the amount of labeled amyloid binding compound bound to the cerebellum of the patient. This ratio is then compared to the same ratio in age-matched normal brain.

Examples of non-aqueous carriers are propylene glycol, polyethylene glycol, vegetable oil, and injectable organic esters such as ethyl oleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobials, anti-oxidants, chelating agents and inert gases. The pH and exact concentration of the various components of the pharmaceutical composition are adjusted according to routine skills in the art.

In an embodiment, a pharmaceutical composition comprising a compound of Formula 1, specifically Formula 1a, is administered to subjects in whom amyloid or amyloid fibril formation are anticipated. In a specific embodiment, such subject is a human and includes, for instance, those who are at risk of developing cerebral amyloid, including the elderly, nondemented population and patients having amyloidosis associated diseases and Type 2 diabetes mellitus. The term “preventing” is intended to include the amelioration of cell degeneration and toxicity associated with fibril formation. By “amelioration” is meant the treatment or prevention of more severe forms of cell degeneration and toxicity in patients already manifesting signs of toxicity, such as dementia.

In another aspect, included is a method of detecting amyloid deposits in biopsy or post-mortem tissue, the method including incubating formalin-fixed tissue with a solution of a compound of Formula 1, specifically Formula 1a. Upon incubation, the compound stains or labels the amyloid deposit in the tissue, and the stained or labeled deposit can be detected or visualized by a standard method. Such detection means include microscopic techniques such as bright-field, fluorescence, laser-confocal and cross-polarization microscopy.

The method of quantifying the amount of amyloid in biopsy or post-mortem tissue involves incubating a labeled amyloid binding compound as disclosed herein, or a water-soluble, non-toxic salt thereof, with a homogenate of biopsy or post-mortem tissue. The tissue is obtained and homogenized by methods well known in the art. A specific label is a radiolabel, although other labels such as enzymes, chemiluminescent and immunofluorescent compounds are well known to skilled artisans. Tissue containing amyloid deposits will bind to the labeled amyloid binding compounds. The bound tissue is then separated from the unbound tissue by a mechanism known to the skilled artisan, such as filtering. The bound tissue can then be quantified through any means known to the skilled artisan. The units of tissue-bound radiolabeled compound are then converted to units of micrograms of amyloid per 100 mg of tissue by comparison to a standard curve generated by incubating known amounts of amyloid with the radiolabeled compound.

The method of distinguishing an Alzheimer's diseased brain from a normal brain involves obtaining tissue from (i) the cerebellum and (ii) another area of the same brain, other than the cerebellum, from normal subjects and from subjects suspected of having Alzheimer's disease. Such tissues are made into separate homogenates using methods well known to the skilled artisan, and then are incubated with a labeled amyloid binding compound. The amount of tissue that binds to the labeled amyloid binding compound is then calculated for each tissue type (e.g., cerebellum, non-cerebellum, normal, abnormal) and the ratio for the binding of non-cerebellum to cerebellum tissue is calculated for tissue from normal and for tissue from patients suspected of having Alzheimer's disease. These ratios are then compared. In one embodiment, if the ratio from the brain suspected of having Alzheimer's disease is above 90% of the ratios obtained from normal brains, the diagnosis of Alzheimer's disease is made. The normal ratios can be obtained from previously obtained data, or alternatively, can be recalculated at the same time the suspected brain tissue is studied.

The novel compounds disclosed herein are novel imaging agents for the imaging of amyloid deposits. Also described are new methods of synthesis of these derivatives, radiolabeling, and methods for diagnosing Alzheimer's disease in vivo by positron emission tomography, magnetic resonance imaging and other imaging methods involving the use of the imaging agents.

The invention is further illustrated by the following non-limiting examples.

### EXAMPLES

Chemicals and Reagents. Common reagents used in the syntheses were purchased from Aldrich Chemical Company, Fluka Chemical Company (Milwaukee, Wis.), Acros (Hampton, N.H.), or Strem Chemicals (Newburyport, Mass.), and were used without further purification, unless otherwise indicated. Water was purified through a Millipore water purification system, comprising a combination of two filters, one Rio™, one reservoir, and one Milli-Q® synthesis system (Bedford, Mass.). Common solvents were obtained from Fisher Scientific (Pittsburgh, Pa.). Brain tissue from deceased Alzheimer's Disease (AD) patients was obtained from Brain Bank of the Clinical Brain Disorders Branch, National Institute of Mental Health, National Institutes of Health.

Instrument and general conditions. Analytical HPLC was performed using a reverse phase column (X-Bridge C18; 10 μm; 10.0×250 mm; Waters) eluted with concentrated ammonia (0.25%) in acetonitrile-water at 6.2 mL/min. The chromatography system was fitted with a continuous wavelength UV-vis detector (Beckman System Gold 168 Detector) and an autosampler (Beckman System Gold 508 Autosampler). For semi-preparative Beckman HPLC, a reverse phase column (X-Bridge™ C18; 10 μm; 19×250 mm; Waters) was eluted at 13 mL/min. The HPLC system was fitted with a manual injector (5 mL injection loop) and a third delivery pump using acetonitrile as eluant at 3 mL/min. The purity of compounds was determined with HPLC monitored for UV absorbance at 350 nm and expressed as area percentage of all peaks. The ¹H and ¹³C NMR spectra of all compounds were acquired on a Bruker DRX 400 (400 MHz ¹H and 100 MHz ¹³C), using the chemical shifts of residual deuterated solvent as the internal standard; chemical shift (δ) data for the proton and carbon resonance were reported in parts per million (ppm) relative to the internal standard. Thin-layer chromatography (TLC) was performed using Silica Gel 60 F254 plates from EM Science and compounds visualized under UV light at either 250 or 360 nm. Flash chromatography was carried out using a Biotage Horizon™ HPFC™ system (Charlottesville, Va., column sizes: 12 mm×150 mm, 25 mm×150 mm, 40 mm×150 mm) with hexanes and ethyl acetate (EtOAc) as eluents with chromatographic solvent proportions expressed on a volume:volume basis. IR spectra were recorded using a Perkin-Elmer Spectrum One FT-IR spectrometer, and UV/vis spectra were recorded using a Lambda 40 UV/vis spectrometer. Mass spectra were acquired using either Thermo Finnigan LCQ^{DECA} LC-MS (MS-HPLC column: Luna C18; 5 μm 2.0×150 mm; Phenomenex, flow rate: 150 μL/min, eluent: MeOH and H₂O mixture) or Thermo Finnigan PolarisQ GC-MS (GC column: capillary RTX-5 ms 30 m×0.25 mm, flow rate: 1 mL/min, carrier gas: He), or VG Micromass 7070E and AutoSpec-Q spectrometers. High-resolution mass spectra (HRMS) were acquired from Mass Spectrometry Laboratory, University of Illinois at Urbana-Champaign (Urbana, Ill.). Elemental analyses of selected compounds were carried out by Midwest Microlab (Indianapolis, Ind.) or Galbraith Laboratories, Inc. (Knoxyille, Tenn.). The melting points were measured using Electrothermal MeI-Temp Manual Melting Point Apparatus (Fisher Scientific), and uncorrected. A CEM Discover microwave system was used for microwave synthesis (Matthews, N.C.).

### Example 1

### Synthesis of Compounds

The following syntheses are illustrated by molecular formulas and reaction schemes, and the structures represented by the acronyms and other symbols used below for the various intermediates and compounds are shown in the Schemes.

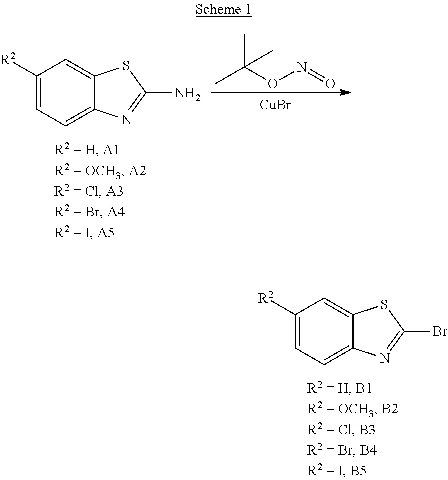

2-Bromo-6-methoxybenzo[d]thiazole (B2) (Scheme 1). 6-Methoxybenzo[d]thiazol-2-amine (5.0 g; 27.7 mmol) was dissolved in 125 mL CH₃CN. t-Butylnitrite (3.4 mL; 28 mmol) was added slowly. CuBr (5.0 g; 34.9 mmol) was added portion-wise through a funnel. The reaction was monitored by HPLC. After 3 hours, ethyl acetate (500 mL) was added, and the mixture was filtered through celite. The organic layer was washed with brine twice (200 mL each). The organic layer was dried over MgSO₄. After filtration, the solvent was removed. The residual was dissolved in 50 mL CH₂Cl₂, and 2 g of silica gel was added. After drying, the silica gel loaded on a silica gel cake in hexane. The product was eluted with 5% ethyl acetate and 95% hexane. A yellowish band was collected (1.5 L). The solvent was removed to afford 0.81 g product. Yield 12%. ¹H NMR (400 MHz; CD₃COCD₃), δ 7.95 (d, ³J_{HH}=8.9 Hz, 1H, Ar—H), 7.61 (d, ⁴J_{HH}=2.6 Hz, 1H, NH), 7.13 (dd, ³J_{HH}=9.0 Hz, ⁴J_{HH}=2.6 Hz, 1H, Ar—H), 3.89 (s, 3H, OCH₃).

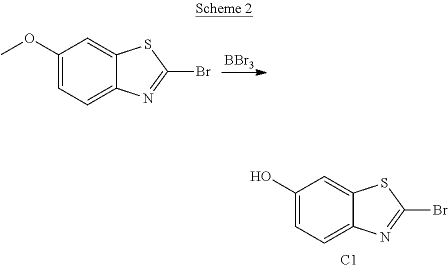

2-Bromobenzo[d]thiazol-6-ol (C1) (Scheme 2). 2-bromo-6-methoxybenzo[d]thiazole (0.90 g; 3.7 mmol) was dissolved in CH₂Cl₂ (50 mL). BBr₃ (1M in CH₂Cl₂, 16 mL, 16 mmol) was added slowly. White precipitate formed immediately. After overnight, HPLC showed complete conversion. Excess CH₂Cl₂ (200 mL) and brine (100 mL) were added. White precipitate formed immediately. The mixture was stirred at RT for 2 hours, until most solid dissolved. The organic layer was separated, and aqueous layer extracted with 2×100 mL CH₂Cl₂. The combined organic solution was dried over MgSO₄. The solvent was removed to afford 0.71 g of product. Yield 83%.

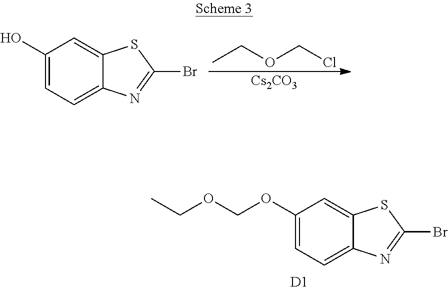

2-Bromo-6-(ethoxymethoxy)benzo[d]thiazole (D1) (Scheme 3). 2-Bromobenzo[d]thiazol-6-ol (0.82 g, 3.6 mmol) was dissolved in 5 mL DMF. Cs₂CO₃ (1.7 g; 5.2 mmol) was added. Chloromethyl ethyl ether (0.5 mL; 5.4 mmol) was added through a syringe. After stirring overnight, HPLC analysis showed a clean product. The reaction mixture was partitioned between ethyl acetate and brine, and extracted with ethyl acetate. The combined ethyl acetate was dried over MgSO₄. After removing solvent, 1.0 g product was obtained as brown oil. Yield 99%. ¹H NMR (400 MHz; CD₃COCD₃), δ 7.96 (s, 1H, Ar—H), 7.93 (d, ³J_{HH}=9.0 Hz, 1H, Ar—H), 7.50 (d, ³J_{HH}=9.0 Hz, 1H, Ar—H), 5.44 (s, 2H, OCH2O), 3.79 (q, ³J_{HH}=7.0 Hz, 2H, OCH₂), 1.18 (t, ³J_{HH}=7.0 Hz, 2H, CH₃).

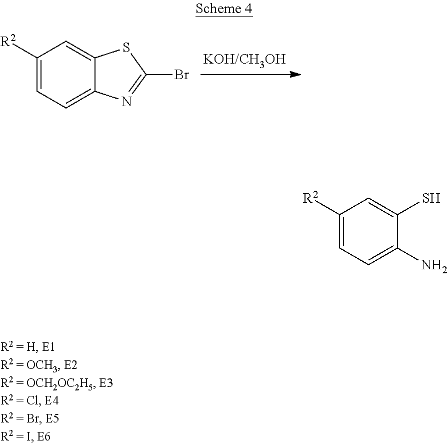

2-Amino-5-(ethoxymethoxy)benzenethiol (E3) (Scheme 4). 2-bromo-6-(ethoxymethoxy)benzo[d]thiazole (1.0 g; 3.5 mmol) was dissolved in 3 mL CH₃OH. KOH (2 g; 35.7 mmol) was added. The mixture was soluble with the help of stiffing and ultrasound. The reaction was subjected to microwave irradiation at 140° C. for 15 minutes at 85 W and 250 psi. HPLC analysis showed complete conversion. An equal volume of ice water was added, and the mixture was cooled in an ice-bath. Acetic acid (3 mL) was added slowly and with shaking. The pH of the solution was monitored to be around 6. The mixture was added into 200 mL brine, and extracted with 3×150 mL ethyl acetate. The combined organic layer was dried over MgSO₄. Removal of solvent afforded a red oil of 0.59 g. Yield 85%. ¹H NMR (400 MHz; CD₃COCD₃), δ 6.90 (dd, ⁴J_{HH}=2.7 Hz, ⁵J_{HH}=0.6 Hz, 1H, Ar—H), 6.88 (dd, ³J_{HH}=8.4 Hz, ⁴J_{HH}=2.8 Hz, 1H, Ar—H), 6.75 (dd, ³J_{HH}=8.4 Hz, ⁵J_{HH}=0.6 Hz, 1H, Ar—H), 5.01 (s, 2H, OCH2O), 3.57 (q, ³J_{HH}=7.0 Hz, 2H, OCH₂), 1.12 (t, ³J_{HH}=7.0 Hz, 2H, CH₃).

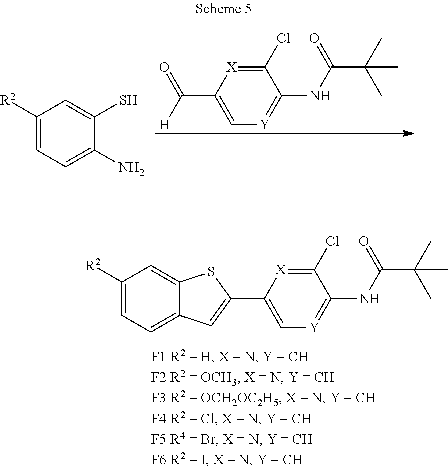

N-(2-chloro-6-(6-(ethoxymethoxy)benzo[d]thiazol-2-yl)pyridin-3-yl)pivalamide (F3) (Scheme 5). 2-Amino-5-(ethoxymethoxy)benzenethiol (0.59 g; 3.0 mmol) and N-(2-chloro-6-formylpyridin-3-yl)pivalamide (0.76 g; 3.2 mmol) were dissolved in 10 mL DMSO. The reaction mixture was subjected to microwave irradiation at 120° C. for 15 minutes at 50 W and 250 psi. HPLC analysis showed complete disappearance of 2-amino-5-(ethoxymethoxy)benzenethiol, but some N-(2-chloro-6-formylpyridin-3-yl)pivalamide remained. Ethyl acetate (300 mL) was added to dissolve the reaction mixture, and the solution was washed by 3×200 mL brine. The collected organic layer was dried over MgSO₄. Removal of solvent afforded 1.1 g of crude product, which was purified by semi-preparative HPLC using 0.025% ammonia and CH₃CN as eluates. After the solvent was removed, the product was obtained at 0.32 g. Yield 25%. ¹H NMR (400 MHz; CD₃COCD₃), δ 8.76 (d, ³J_{HH}=8.4 Hz, 1H, Ar—H), 8.32 (d, ³J_{HH}=8.4 Hz, 1H, Ar—H), 7.97 (d, ³J_{HH}=8.8 Hz, 1H, Ar—H), 7.75 (d, ⁴J_{HH}=2.4 Hz, 1H, Ar—H), 7.25 (dd, ³J_{HH}=8.9 Hz, ⁴J_{HH}=2.4 Hz, 1H, Ar—H), 5.36 (s, 2H, OCH2O), 3.76 (q, ³J_{HH}=7.1 Hz, 2H, OCH₂), 1.38 (s, 9H, CH₃), 1.19 (t, ³J_{HH}=7.1 Hz, 3H, CH₃).

N-(2-chloro-6-(6-methoxybenzo[d]thiazol-2-yl)pyridin-3-yl)pivalamide (F2) (Scheme 5). 2-Amino-5-(methoxy)benzenethiol (0.073 g; 0.47 mmol) and N-(2-chloro-6-formylpyridin-3-yl)pivalamide (0.100 g; 0.42 mmol) were dissolved in 2 mL DMSO. The reaction mixture was subjected to microwave irradiation at 120° C. for 10 minutes at 30 W and 250 psi. HPLC analysis showed pure product. Ethyl acetate (100 mL) was added to dissolve the reaction mixture, and the solution was washed by 3×200 mL brine. The collected organic layer was dried over MgSO₄. After removing the solvent, the crude product was washed by hexane. After the solvent was removed, the product was obtained at 0.096 g. Yield 63%. ¹H NMR (400 MHz; CDCl₃), δ 8.95 (d, ³J_{HH}=8.5 Hz, 1H, Ar—H), 8.52 (d, ³J_{HH}=8.5 Hz, 1H, Ar—H), 8.14 (brs, 1H, NH), 8.07 (d, ³J_{HH}=9.0 Hz, 1H, Ar—H), 7.35 (d, ⁴J_{HH}=2.4 Hz, 1H, Ar—H), 7.14 (dd, ³J_{HH}=9.0 Hz, ⁴J_{HH}=2.5 Hz, 1H, Ar—H), 3.90 (s, 3H, OCH₃), 1.36 (s, 9H, CH₃).

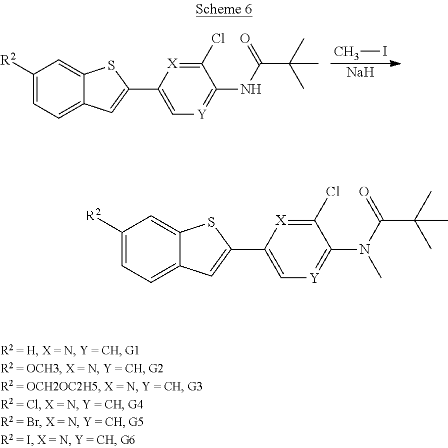

N-(2-chloro-6-(6-(ethoxymethoxy)benzo[d]thiazol-2-yl)pyridin-3-yl)-N-methylpivalamide (G3) (Scheme 6). N-(2-chloro-6-(6-(ethoxymethoxy)benzo[d]thiazol-2-yl)pyridin-3-yl)pivalamide (0.32 g; 0.76 mmol) was dissolved in 20 mL THF. NaH (50 mg; 2.1 mmol), and CH₃I (135 μL, 2.2 mmol) were added. After overnight stiffing, HPLC analysis showed complete conversion. The mixture was partitioned between ethyl acetate and brine, and the aqueous phase was extracted with ethyl acetate 2×100 mL. The combined organic phase was dried over MgSO₄. After filtration, the solvent was removed to afford 0.31 g product. Yield 94%. ¹H NMR (400 MHz; CD₃COCD₃), δ 8.40 (d, ³J_{HH}=8.0 Hz, 1H, Ar—H), 8.15 (d, ³J_{HH}=8.0 Hz, 1H, Ar—H), 8.01 (d, ³J_{HH}=9.0 Hz, 1H, Ar—H), 7.79 (d, ⁴J_{HH}=2.4 Hz, 1H, Ar—H), 7.28 (dd, ³J_{HH}=9.0 Hz, ⁴J_{HH}=2.4 Hz, 1H, Ar—H), 5.38 (s, 2H, OCH2O), 3.77 (q, ³J_{HH}=7.1 Hz, 2H, OCH₂), 3.27 (s, 3H, NCH3), 1.19 (t, ³J_{HH}=7.1 Hz, 3H, CH₃), 1.15 (s, 9H, CH₃).

N-(2-chloro-6-(6-methoxybenzo[d]thiazol-2-yl)pyridin-3-yl)-N-methylpivalamide (G2) (Scheme 6). N-(2-chloro-6-(6-methoxybenzo[d]thiazol-2-yl)pyridin-3-yl)pivalamide (0.17 g; 0.45 mmol) was dissolved in 5.0 mL THF. NaH (30 mg; 1.25 mmol) and CH₃I (0.2 mL; 3.2 mmol) were added. After overnight stiffing, HPLC analysis showed complete conversion. The mixture was partitioned between ethyl acetate and brine. The aqueous phase was extracted with ethyl acetate 2×100 mL. The combined organic phase was dried over MgSO₄. After filtration, the solvent was removed to afford 0.16 g product. Yield 91%. ¹H NMR (400 MHz; CDCl₃), δ 8.28 (d, ³J_{HH}=8.5 Hz, 1H, Ar—H), 7.96 (d, ³J_{HH}=8.5 Hz, 1H, Ar—H), 7.73 (d, ³J_{HH}=9.0 Hz, 1H, Ar—H), 7.38 (d, ⁴J_{HH}=2.4 Hz, 1H, Ar—H), 7.11 (dd, ³J_{HH}=9.0 Hz, ⁴J_{HH}=2.5 Hz, 1H, Ar—H), 3.89 (s, 3H, OCH₃), 3.21 (s, 3H, NCH₃), 1.13 (s, 9H, CH₃).

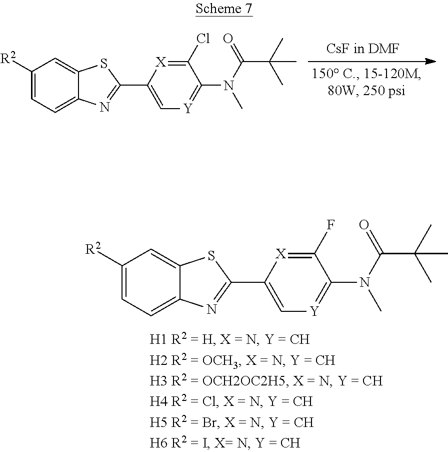

N-(6-(6-(ethoxymethoxy)benzo[d]thiazol-2-yl)-2-fluoropyridin-3-yl)-N-methylpivalamide (H3) (Scheme 7). N-(2-chloro-6-(6-(ethoxymethoxy)benzo[d]thiazol-2-yl)pyridin-3-yl)-N-methylpivalamide (0.022 g; 0.051 mmol) was dissolved in 0.5 mL DMF. CsF (62 mg; 0.41 mmol) was added. The reaction mixture was subjected to microwave irradiation at 150° C. for 120 minutes at 80 W and 250 psi. HPLC analysis showed complete conversion of the starting material.

N-(2-fluoro-6-(6-methoxybenzo[d]thiazol-2-yl)pyridin-3-yl)-N-methylpivalamide (H2) (Scheme 7). N-(2-chloro-6-(6-methoxybenzo[d]thiazol-2-yl)pyridin-3-yl)-N-methylpivalamide (0.17 g; 0.44 mmol) was dissolved in 3.0 mL of DMF in 35 mL microwave tube with a stir-bar. CsF (2 g; 13 mmol) was added. The mixture was subjected to microwave irradiation at 150° C. for 60 minutes at 80 W and 250 psi. HPLC analysis showed complete conversion. The mixture was filtered, and the product purified by semi-preparative HPLC. ¹H NMR (400 MHz; CDCl₃), δ 8.23 (dd, ³J_{HH}=7.9 Hz, ⁵J_{HF}=0.8 Hz, 1H, Ar—H), 7.95 (d, ³J_{HH}=9.0 Hz, 1H, Ar—H), 7.79 (dd, ⁴J_{HF}=9.2 Hz, ³J_{HH}=8.0 Hz, 1H, Ar—H), 7.38 (d, ⁴J_{HH}=2.5 Hz, 1H, Ar—H), 7.12 (dd, ³J_{HH}=9.0 Hz, ⁴J_{HH}=2.6 Hz, 1H, Ar—H), 3.90 (s, 3H, OCH₃), 3.23 (s, 3H, NCH₃), 1.13 (s, 9H, CH₃).

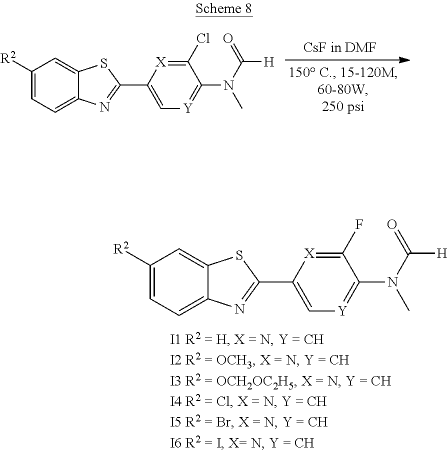

N-(6-(6-(Ethoxymethoxy)benzo[d]thiazol-2-yl)-2-fluoropyridin-3-yl)-N-methylformamide (I3) (Scheme 8). N-(2-chloro-6-(6-(ethoxymethoxy)benzo[d]thiazol-2-yl)pyridin-3-yl)-N-methylformamide (22.1 mg; 0.058 mmol) was dissolved in 0.5 mL DMF, and CsF (62 mg; 0.41 mmol) was added. The mixture was subjected to microwave irradiation at 150° C., for 120 minutes at 60 W power and a pressure of 250 pounds per square inch (psi). HPLC analysis showed complete conversion.

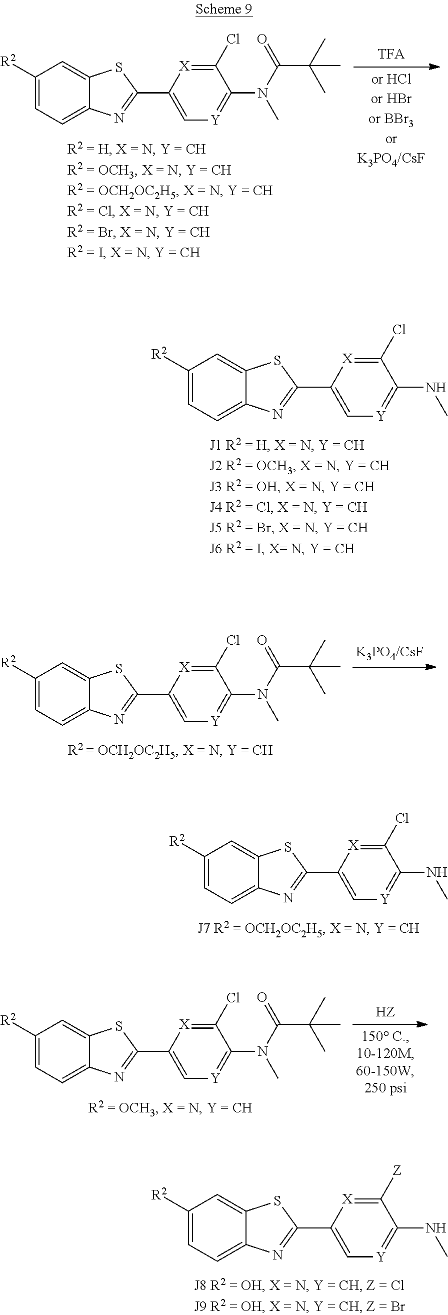

2-Chloro-6-(6-methoxybenzo[d]thiazol-2-yl)-N-methylpyridin-3-amine (J2) (Scheme 9). N-(2-chloro-6-(6-methoxybenzo[d]thiazol-2-yl)pyridin-3-yl)-N-methylpivalamide (10 mg; 0.026 mmol) was dissolved in 0.5 mL DMF. CsF and K₃PO₄ (1:1 by weight, 50 mg) were added. The mixture was subjected to microwave irradiation at 150° C., for 120 minutes at 60 Watt (W) power and a pressure of 250 pounds per square inch. HPLC analysis showed complete conversion. The mixture was partitioned between ethyl acetate and water. The combined organic layer was dried over MgSO₄. After the solvent was removed, the crude product was purified by semi-preparative HPLC purification to generate 6 mg product. Yield 76%. ¹H NMR (400 MHz; CDCl₃), δ 8.14 (d, ³J_{HH}=8.1 Hz, 1H, Ar—H), 7.96 (d, ³J_{HH}=9.4 Hz, 1H, Ar—H), 7.36 (d, ⁴J_{HH}=2.6 Hz, 1H, Ar—H), 7.07 (dd, ³J_{HH}=8.5 Hz, ⁴J_{HH}=2.6 Hz, 1H, Ar—H), 6.97 (d, ³J_{HH}=8.1 Hz, 1H, Ar—H), 3.90 (s, 3H, OCH₃), 3.00 (d, ³J_{HH}=5.1 Hz, 3H, NCH₃).

2-(6-Chloro-5-(methylamino)pyridin-2-yl)benzo[d]thiazol-6-ol (J8) (Scheme 9). N-(2-chloro-6-(6-methoxybenzo[d]thiazol-2-yl)pyridin-3-yl)-N-methylpivalamide (0.17 g; 0.44 mmol) was dissolved in 6 mL concentrated HCl, and the mixture was subjected to microwave irradiation at 150° C. for 15 minutes at 150 W and 250 psi. HPLC analysis showed complete conversion. The mixture was quenched using NaHCO₃ until neutral, and the product was extracted using ethyl acetate. The combined organic layer was dried over MgSO₄. After the solvent was removed, the crude product was obtained at 121 mg. Semi-preparative HPLC purification generated 42.5 mg product. Yield 34%.

2-(6-Bromo-5-(methylamino)pyridin-2-yl)benzo[d]thiazol-6-ol (J9) (Scheme 9). N-(2-chloro-6-(6-methoxybenzo[d]thiazol-2-yl)pyridin-3-yl)-N-methylpivalamide (10 mg; 0.026 mmol) was dissolved in 3 mL concentrated HBr, and the mixture was subjected to microwave irradiation at 120° C. for 10 minutes at 80 W and 250 psi. HPLC analysis showed complete conversion. The mixture was quenched using NaHCO₃ until neutral, and the product was extracted using ethyl acetate. The combined organic layer was dried over MgSO₄. After the solvent was removed, the crude product was purified by semi-preparative HPLC purification to generate 7 mg product. Yield 81%.

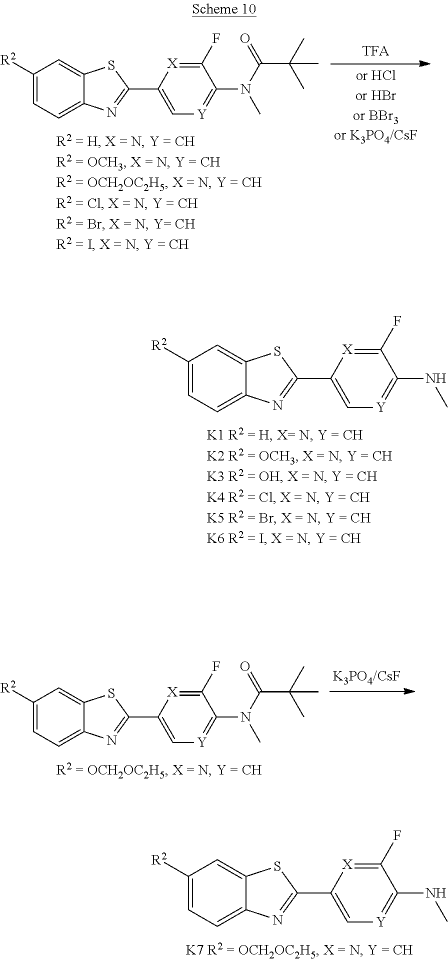

2-Fluoro-6-(6-methoxybenzo[d]thiazol-2-yl)-N-methylpyridin-3-amine (K2) (Scheme 10). N-(2-fluoro-6-(6-methoxybenzo[d]thiazol-2-yl)pyridin-3-yl)-N-methylpivalamide (10 mg; 0.026 mmol) was dissolved in 0.5 mL 48% HBr. The mixture was placed in an ultrasonic bath to dissolve. The color of the solution changed from yellow to yellow greenish. HPLC analysis showed the reaction was complete. ¹H NMR (400 MHz; CDCl₃), δ 8.08 (d, ³J_{HH}=8.2 Hz, 1H, Ar—H), 7.86 (d, ³J_{HH}=9.0 Hz, 1H, Ar—H), 7.34 (d, ⁴J_{HH}=2.6 Hz, 1H, Ar—H), 7.05 (dd, ³J_{HH}=9.0 Hz, ⁴J_{HH}=2.6 Hz, 1H, Ar—H), 7.00 (dd, ⁴J_{HF}=10.3 Hz, ⁴J_{HH}=2.6 Hz, 1H, Ar—H), 3.87 (s, 3H, OCH₃), 2.96 (d, ³J_{HH}=5.3 Hz, 3H, NCH₃).

2-(6-Fluoro-5-(methylamino)pyridin-2-yl)benzo[d]thiazol-6-ol (K3) (Scheme 10). N-(2-chloro-6-(6-(ethoxymethoxy)benzo[d]thiazol-2-yl)pyridin-3-yl)-N-methylpivalamide (20 mg; 0.046 mmol) was dissolved in 0.5 mL TFA. The mixture was subjected to microwave irradiation at 100° C. for 5 minutes at 30 W and 250 psi. HPLC analysis showed complete conversion. The mixture was diluted with 0.5 mL DMF. The product was purified using semi-preparative HPLC. ¹H NMR (400 MHz; DMF-d₇), δ 8.07 (d, ³J_{HH}=8.2 Hz, 1H, Ar—H), 7.77 (d, ³J_{HH}=9.8 Hz, 1H, Ar—H), 7.47 (d, ⁴J_{HH}=2.7 Hz, 1H, Ar—H), 7.24 (dd, ⁴J_{HF}=11.8 Hz, ³J_{HH}=9.0 Hz, 1H, Ar—H), 7.05 (dd, ³J_{HH}=9.8 Hz, ⁵J_{HF}=3.1 Hz, 1H, Ar—H), 2.95 (d, ³J_{HH}=5.3 Hz, 3H, NCH₃).

2-(6-Fluoro-5-(methylamino)pyridin-2-yl)benzo[d]thiazol-6-ol (K3) (Scheme 10). N-(2-chloro-6-(6-methoxybenzo[d]thiazol-2-yl)pyridin-3-yl)-N-methylpivalamide (20 mg; 0.046 mmol) was dissolved in 0.5 mL 48% HBr. The mixture was subjected to microwave irradiation at 120° C. for 5 minutes at 30 W and 250 psi. HPLC analysis showed complete conversion. The mixture was diluted with 0.5 mL DMF. The product was purified using semi-preparative HPLC.

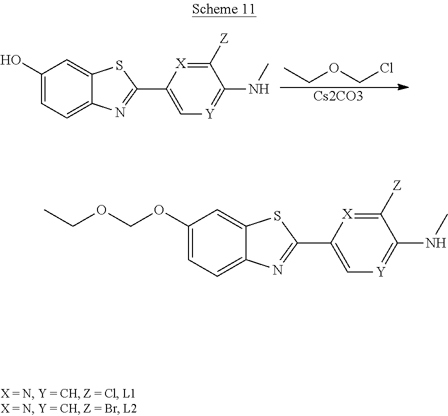

2-Chloro-6-(6-(ethoxymethoxy)benzo[d]thiazol-2-yl)-N-methylpyridin-3-amine (L1) (Scheme 11). 2-(6-Chloro-5-(methylamino)pyridin-2-yl)benzo[d]thiazol-6-ol (42.5 mg; 0.15 mmol) was dissolved in 5 mL of DMF in round flask with an equal pressure addition funnel. ClCH₂OC₂H₅ (16 μL, 0.18 mmol) was dissolved in 5 mL THF. The solution was placed in the addition funnel. The setup was transferred out of the glove box, and cooled in a dry-ice/ethanol bath. ClCH₂OC₂H₅ solution was added dropwise. After addition, the solution was left to warm up to RT. After stiffing overnight, HPLC analysis showed complete conversion. The mixture was partitioned between ethyl acetate and brine. The combined organic layer was dried over MgSO₄. The solvent was removed to afford the product.

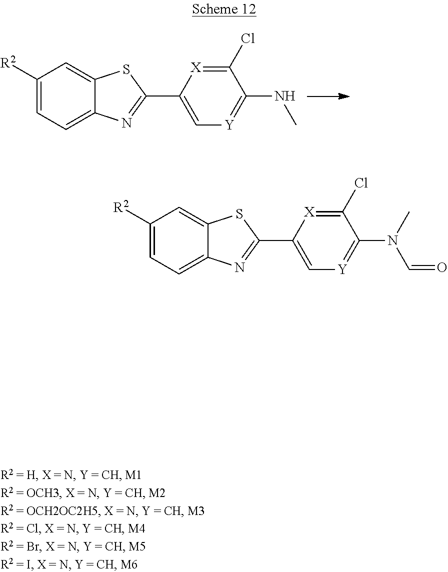

2N-(2-chloro-6-(6-(ethoxymethoxy)benzo[d]thiazol-2-yl)pyridin-3-yl)-N-methylformamide (M3) (Scheme 12). Equal volumes of formic acid and acetic anhydride were microwaved at 60° C. for 15 min. The mixture anhydride (100 μL) was added to a solution of 2-Chloro-6-(6-(ethoxymethoxy)benzo[d]thiazol-2-yl)-N-methylpyridin-3-amine (42 mg; 0.12 mmol) dissolved in 5 mL DMF. HPLC analysis showed complete reaction. The mixture was partitioned between ethyl acetate and brine. The combined organic layer was dried over MgSO₄. The solvent was removed to afford 48 mg of product. Yield 87%. Radiolabeling.

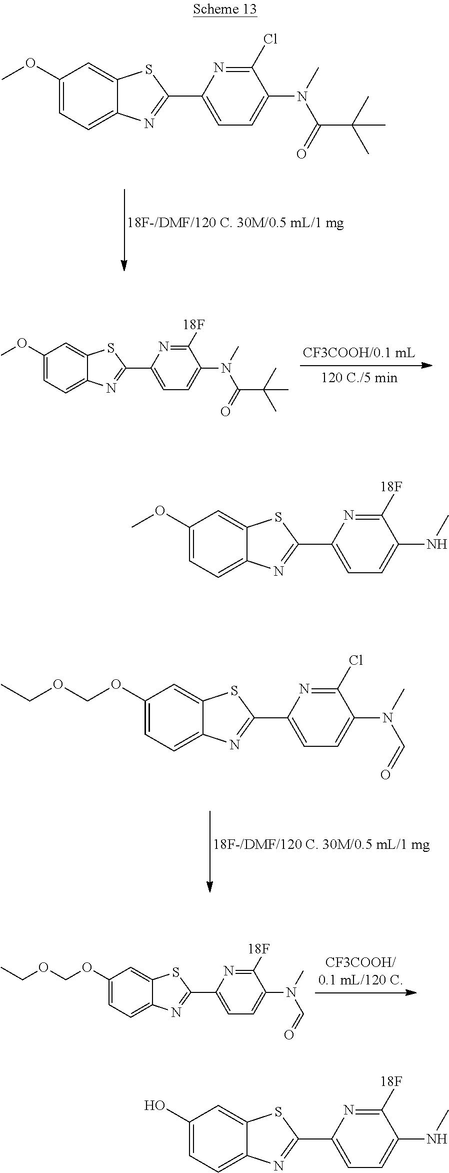

A process for radiolabeling the methyl and the —CH₂OCH₂CH₃ derivatives is shown in Scheme 13. Radiolabeling can proceed through the pivaloyl intermediate or the formyl intermediate.

### Example 2

### Synthesis of Compounds 1.4, 1.5 and 1.6

A synthesis scheme for compounds 1.4, 1.5, and 1.6 is shown in FIG. 2. The precursors for radiolabeling were synthesized in 8-steps (Experimental). [¹⁸F]FNIMHa-c (compounds 1.6, 1.5, and 1.4) were synthesized by nucleophilic substitution in aryl chlorides in high radiochemical yield (30-36% decay-corrected). The Boc protecting group was removed under acid conditions at high temperature.

### Example 3

### In Vitro Binding Assay

### Protocol

AD brain tissue was homogenized at 1:500 in PBS, and 800 μL suspension was used in each tube. [³H]6-OH-BTA-1 with a concentration of 1 mCi/mL stock solution was diluted using ethanol to give an intermediate solution of 1 μCi/100 μL, which was further diluted using PBS to result in a dilute stock solution of 2.7×10⁻² μCi/100 μL, and 100 μL was used in each tube (2 vials with 100 μL of [³H]6-OH-BTA-1 each and scintillation fluid as references). Cold 6-OH-BTA-1 or other displacer was dissolved in ethanol to result in a stock solution of 1×10⁻³ M, which was further diluted using PBS or ethanol to result in solutions with concentrations ranging from 1×10⁻⁵ to 10⁻¹⁰ M, and 100 μL was used in each tube. After assembly, the tubes were vortexed, and incubated for 3 hrs at room temperature. After separation using a cell harvester, the filter paper (GF/B filter paper pretreated with 0.5% polyimine solution) was washed with 10% ethanol in PBS (3×3 mL). The filters were placed into 20 mL glass vials and 10 mL scintillation fluid each was added. After overnight incubation, the samples were counted. The data were analyzed using GraphPad Prism 4 or KaleidaGraph 3.6.

Results of In Vitro Assay of benzo[d]thiazole Derivatives.

Determination of the binding affinities of new ligands for β-amyloid is the first step in selecting candidate radioligands for PET studies in humans. Three types of amyloid plaques have been used to assay ligand binding in vitro, namely synthetic aggregates of Aβ₁₋₄₀, Aβ₁₋₄₂, amyloid plaques from transgenic mice, and amyloid plaques from human Alzheimer's disease (AD) brain tissue. Results from the three types and also from different batches of synthetic amyloid plaques vary with respect to binding site concentration. These findings may reflect variations in binding site architecture, though no significant difference in ligand binding affinity has been detected among the three types of amyloid plaques for the binding site typical of 6-OH-BTA-1. Since the ultimate test for a radioligand is successful application in humans, in vitro evaluation using human AD brain tissue is highly appropriate. However, it should be noted that other proteins with similar binding sites might interfere with the binding of the ligand to β-amyloid plaques. This may then be reflected in a lower percentage of displaceable radioactivity in the binding assay. The use of isolated human amyloid plaques may also help to identify other native binding sites.

Isolated plaques were used in developing an in vitro binding assay. Tritiated 6-OH-BTA-1 was selected as the reference radioligand based on its use in human PET imaging and high affinity. Displacement curves were created using non-radioactive 6-OH-BTA-1 and other novel ligands.

The displacement of [³H]6-OH-BTA-1 by non-radioactive 6-OH-BTA-1 or the test ligands resulted in classical displacement curves. Some lipophilic ligands required ethanol in the medium to increase their solubility and to effect displacement of reference radioligand. Non-radioactive 6-OH-BTA-1 achieved >95% displacement of reference radioligand, indicating that the presence of competing binding sites for the isolated amyloid plaques was negligible. This assay could therefore be used to screen compounds for amyloid binding without interference from other proteins, such as tau-tangles. This displacement curve was also analyzed using a homologous displacement mathematical model to extract the Bₘₐₓ of the amyloid plaques. The measured Bₘₐₓ is linear with the amount of amyloid used in the experiment. The ratio between Bₘₐₓ and the amount of Aβ₁₋₄₂ monomer measured by ELISA is about 1:2, somewhat less than that reported previously. The denaturing agents used to aid dissolution of the amyloid plaques may account for the observed difference.

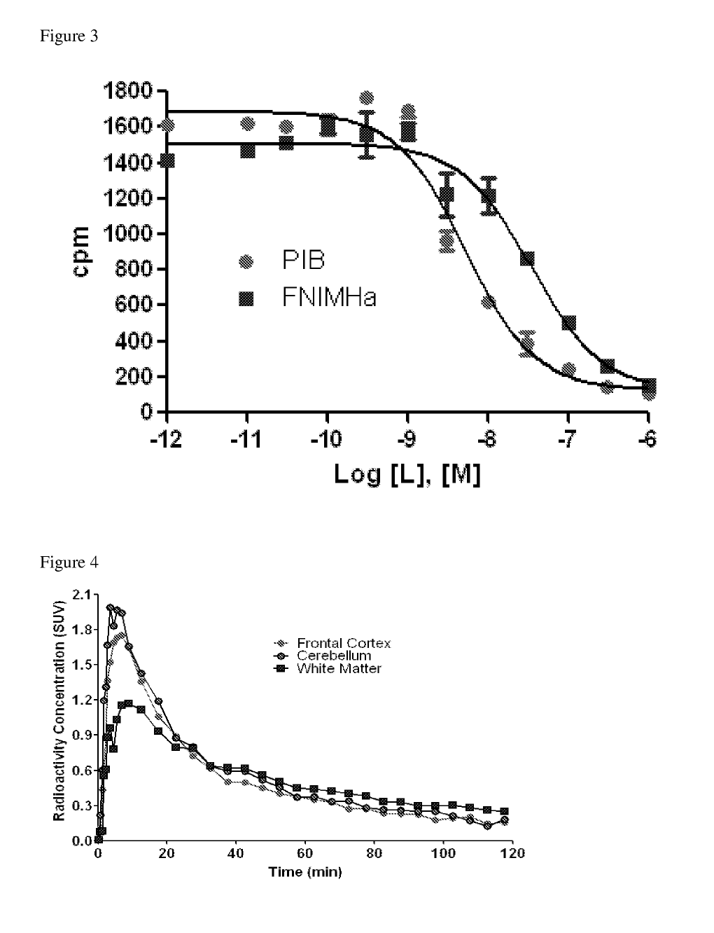

The binding affinities for a variety of benzo[d]thiazole derivatives of Formula I are listed in Table 2. In this table, Ki is a measure (in nM units) of the binding affinity of the compound toward beta-amyloid plaques in AD brain tissue, measured through a competitive radioligand displacement in vitro assay using [³H]6-OH-BTA-1 as the reference radioligand. The lipophilicity of the compound is represented by c Log D_{7.4} which is calculated through a software package (ACD/Log D version 8.0) at pH=7.4, a close mimic of physiological conditions. Ki was measured for compounds 1.4, 1.5, and 1.6. The binding curves for compound 1.6 and [¹¹C]PIB are given in FIG. 3 and the Ki's are in Table 2.

TABLE 2
Compounds tested for binding to amyloid plaques.

| Ligand | R¹ | Y | X | R² | cLogD_{7.4} | Ki (nM) |
|---|---|---|---|---|---|---|
| 1.1 | H | CH | N | H | 3.56 | |
| 1.2 | H | CH | N | OCH₃ | 3.47 | |
| 1.3 | H | CH | N | OH | 2.75 | |
| 1.4 | CH₃ | CH | N | H | 2.74 | 5 |
| 1.5 | CH₃ | CH | N | OCH₃ | 2.66 | 10 |
| 1.6 | CH₃ | CH | N | OH | 1.94 | 20 |
| 1.7 | CH₂CH₂ | CH | N | H | 2.77 | |
| 1.8 | CH₂CH₂ | CH | N | OCH₃ | 2.69 | |
| 1.9 | CH₂CH₂ | CH | N | OH | 1.97 | |
| 1.10 | CH₂CH₂CH₂ | CH | N | H | 3.44 | |
| 1.11 | CH₂CH₂CH₂ | CH | N | OCH₃ | 3.35 | |
| 1.12 | CH₂CH₂CH₂ | CH | N | OH | 2.63 | |
| 1.13 | CH₂S | CH | N | H | 2.57 | |
| 1.14 | CH₂S | CH | N | OCH₃ | 2.49 | |
| 1.15 | CH₂S | CH | N | OH | 1.77 | |
| 1.16 | CH₂CH₂S | CH | N | H | 4.24 | |
| 1.17 | CH₂CH₂S | CH | N | OCH₃ | 4.15 | |
| 1.18 | CH₂CH₂S | CH | N | OH | 3.43 | |

### Example 4

### Evaluation of Radioligands in Monkey In Vivo with PET

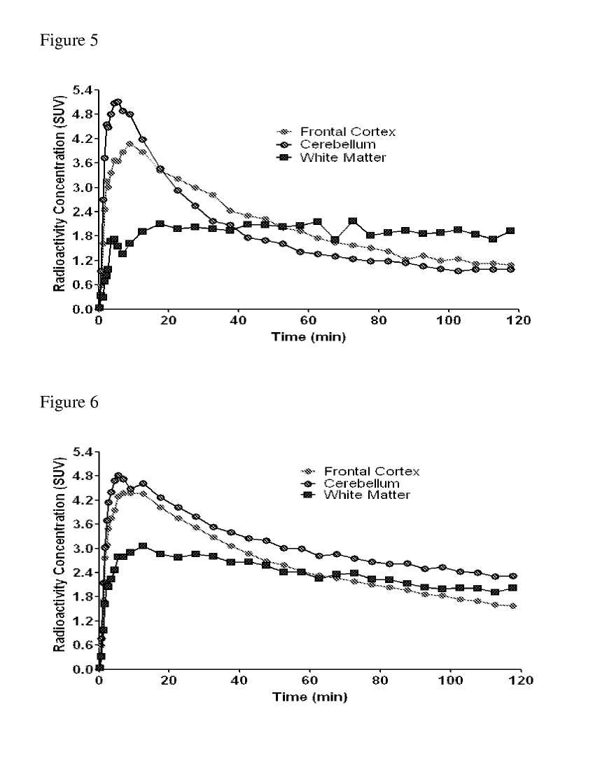

A male rhesus monkey (15 kg) was initially immobilized with ketamine (15 mg/kg) and subsequently anesthetized with isoflurane (1.5%) for the duration of the experiment. The monkey was placed prone in the PET camera (HRRT, Siemens/CPS, Knoxyille, Tenn., USA). A fixation device was used to secure the monkey's head during scanning. A urinary catheter was inserted and clamped so that the activity overlaying the bladder represented the total urinary excretion during the scan. Electrocardiogram, body temperature, heart and respiration rates were measured throughout the experiment. Body temperature was controlled and monitored by a forced-air temperature management unit (Bair Hugger Model 505; Arizant Healthcare Inc.; MN, USA). The scanner consisted of eight flat panel detectors with a transaxial and axial coverage of 31.2 cm and 25.2 cm, respectively. The scanner is also equipped with dual-layered phoswich detector allowing depth-of-interaction. Dynamic PET scans were acquired in 64-bit list mode format, following the intravenous administration of Compound 1.6 (2.9 mCi). The scan lasted for 2 hours containing 33 frames with duration ranging from 30 seconds to 5 minutes. Data were reconstructed into a 256×256×207 image matrix (voxel size 1.21×1.21×1.23 mm), using a 3D list mode OSEM algorithm. The reconstructed image resolution was 2.5 mm. Transmission scan was acquired with a rotating ¹³⁷Cs point source for 6 minutes and used to correct for attenuation. A model-based scatter correction was applied. Tomographic images were analyzed with PMOD 2.6. Time activity curves (FIGS. 4-6) were calculated in % SUV for volume of interest (VOIs), defined by co-registration with MRI (see below), and compared for uptake and washout between different brain regions.

Co-registration of PET Data with MRI. All frames of the original reconstructed PET data were summed and then co-registered to a T₁-weighted magnetic resonance (MR) image acquired separately on a 1.5 T Signa MR scanner (GE Medical Systems) with image analysis software MEDx (Sensor Systems Inc.; Sterling, Va., U.S.A.). The summed PET image was fused with the co-registered MR image with an image fusion tool in PMOD. Several VOIs for the source organs were then manually defined on this fused image with anatomical structures identified on the MR image.

In normal male rhesus monkeys, [¹⁸F]FNIMIHa-c (compounds 1.6, 1.5 and 1.4) entered the brain very well and washed out fast (FIGS. 4-6, respectively). Among the three radioligands evaluated, FNIMHa showed the most promising pharmacokinetics.

### Example 5

### Autoradiography of Compound 1.6 to Post Mortem Human Brain Tissue

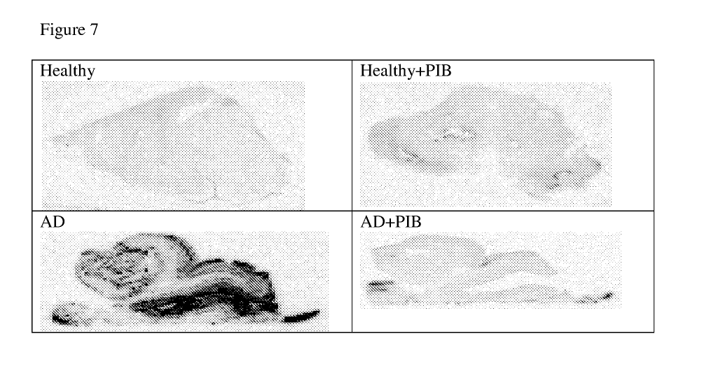

Coronal sections of cerebrum from a confirmed case of AD (female, 59 year old, postmortem interval (PMI) 47.5 hours) and a normal control (female, 64 years old, PMI 28.5 hours) were frozen rapidly in an equal mixture of dry ice and isopentane, sealed in a plastic bag and stored at −76° C. before sectioning. Frozen blocks of the medial temporal lobe (hippocampal region) were sectioned at a thickness of 14 μm, mounted on gelatin-coated glass slides, dried and stored under desiccant at −76° C. Before use, slide-mounted tissue sections were removed from the freezer, thawed at room temperature for 20 min, and air-dried. Compounds 1.6, 1.5 and 1.4 (1.0 mg) were dissolved in DMSO (3.529 mL) resulting in a stock solution of 1 mM, which was then further diluted with DMSO to 0.1 mM and then diluted with PBS to 1 μM. The AD tissue and normal tissue slides were pretreated with either a stock solution of Compounds 1.6, 1.5, and 1.4 or PBS for 20 min at room temperature. All slides were incubated for 20 min at room temperature in [¹¹C]PIB formulation solution (0.3 mCi). They were then dipped in ethanol for 5 min. The slides were dried on a hot plate with a stream of cold air and placed in a cassette with the exposed sides facing up. The phosphor imaging plate was held with the blue side down. The entire cassette was placed in the dark at room temperature overnight for ¹¹C-labeling. Digital autoradiography was acquired using a FUJI BAS 5000 phosphorimager (FUJI, Tokyo, Japan) with a resolution of 25 μm.

[¹⁸F]FNIMHa-c (compounds 1.6, 1.5, and 1.4) showed clear displaceable binding in AD brain tissue slides (FIG. 7) in amyloid-loaded gray matter region, while no radioactivity was observed on healthy brain tissue. The specific signal is displaceable by ligand itself or PIB.

In summary, three compounds of formula 1 were prepared and evaluated in normal monkey. All three compounds showed high initial uptake in brain and fast washout afterwards. [¹⁸F]FNIMIHa (compound 1.6) showed uniform distribution of radioactivity at less than 1 hour of injection and specific binding with amyloid plaques from autoradiography.

The terms “a” and “an” do not denote a limitation of quantity, but rather denote the presence of at least one of the referenced items. The term “or” means “and/or.” The terms “comprising”, “having”, “including”, and “containing” are to be construed as open-ended terms (i.e., meaning “including, but not limited to”). Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Unless defined otherwise, all technical and scientific terms used herein have the same meaning as is commonly understood by one of skill in the art to which this invention belongs.

All compounds are understood to include all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

Compounds are described using standard nomenclature. The term “substituted” means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example, a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent.

“Alkyl” means a branched or straight chain saturated aliphatic hydrocarbon group having the specified number of carbon atoms. The term C₁-C₆ alkyl indicates an alkyl group having 1, 2, 3, 4, 5, or 6 carbon atoms. Examples of alkyl include, but are not limited to, methyl, ethyl, n-propyl, isopropyl, n-butyl, 3-methylbutyl, t-butyl, n-pentyl, and sec-pentyl.

“Alkylene” means a branched or straight chain saturated aliphatic hydrocarbon group having the specified number of carbon atoms and a valence of two. Examples of alkylene groups include, but are not limited to, methylene (—(CH₂)—) and (ethylene —(CH₂)₂—).

“Alkylthio” means an alkyl group as defined above attached through a sulfur linkage, i.e., a C₂ alkylthio is a group of the formula CH₃CH₂S—.

“Thio-C₁-C₃ alkylene” means an alkylene group containing a sulfur linkage either internally or at a terminus. For example, a thio-C₁-alkylene is a group of the formula —S—CH₂ and a thio-C₂-alkylene is a group of the formula —S—CH₂CH₂— or —CH₂—S—CH₂—.

“Alkenyl” means a branched or straight chain aliphatic hydrocarbon group having the specified number of carbon atoms and at least one carbon-carbon double bond. For example, an ethenyl group is a C2 alkenyl group of the formula CH₂═CH—.

“Alkenylene” means a branched or straight chain aliphatic hydrocarbon group having the specified number of carbon atoms and at least one carbon-carbon double bond, and a valence of two. For example, an ethenylene group is a C2 alkenylene group of the formula —CH═CH—.

“Hydroxyalkyl” means both branched and straight-chain alkyl groups having the specified number of carbon atoms, substituted with one or two hydroxyl (—OH) groups. The hydroxyl group(s) can be located anywhere on the chain that allows for substitution.

“Alkoxy” means an alkyl group, as defined above, with the indicated number of carbon atoms attached via an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Haloalkyl” means both branched and straight-chain alkyl groups having the specified number of carbon atoms, substituted with one or more halogen atoms (F, Cl, Br, I, or As), generally up to the maximum allowable number of halogen atoms allowed by the valence of the alkyl group being substituted. Examples of haloalkyl include, for example, trifluoromethyl, difluoromethyl, 2-fluoroethyl, and penta-fluoroethyl. “Haloalkoxy” means a haloalkyl group as defined above attached through an oxygen bridge, e.g., —OCF₃.

“Aryl” means an aromatic group containing only carbon in the aromatic ring or rings. Such aromatic groups may be further substituted with carbon or non-carbon atoms or groups. Aryl groups can contain one or two separate, fused, or pendant rings and from 6 to about 12 ring atoms, without heteroatoms as ring members. Aryl groups include, for example, phenyl, naphthyl, including 1-naphthyl and 2-naphthyl, and bi-phenyl. “Alkylthio” means an aryl group bound to the group it substitutes through a sulfur linkage, i.e., a C6 phenylthio is a group of the formula C₅H₅S—.

“Arylalkylene” means an alkylene group having an aryl substituent as described above, and the number of indicated number of carbon atoms in total. For example, a benzyl group is a C7 arylalkylene group.

“Carbamoyl C1-C6 alkyl” is an alkyl group covalently bonded to —C(O)NH2, e.g., —C(O)NHCH3 is a carbamoyl C1 alkyl.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like.

The term “carrier” applied to pharmaceutical compositions of the disclosure refers to a diluent, excipient, or vehicle with which an active compound is provided. An excipient is an inactive ingredient useful in preparing a pharmaceutical composition that is generally safe, non-toxic, and neither biologically nor otherwise undesirable, and includes an excipient that is acceptable for veterinary use as well as human pharmaceutical use.

All references cited herein are incorporated by reference in their entirety.

While specific embodiments have been shown and described, various modifications and substitutions can be made thereto without departing from the spirit and scope of the invention. Accordingly, it is to be understood that the present invention has been described by way of illustration and not limitations.

1. A compound having Formula 1 or a pharmaceutically acceptable salt thereof. 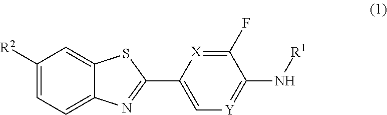
wherein
X is CH or N;
Y is CH or N;
wherein X and Y are not the same;
R¹ is H, C₁-C₆ alkyl, C₁-C₆ haloalkyl, C₁-C₆ hydroxyalkyl, C₇-C₁₃ arylalkylene, carbamoyl C₁-C₆ alkyl, or Y and R¹ are joined to form a C₂-C₄ alkylene, C₂-C₄ alkenylene, or thio-C₁-C₃ alkylene linkage between Y and the amine nitrogen to which R¹ is attached; and
R² is H, OH, halo, C₁-C₆ alkyl, C₁-C₆ haloalkyl, C₁-C₆ alkoxy, C₁-C₆ haloalkoxy, C₁-C₆ alkylthio, or C₆-C₁₂ aryl.

2. The compound of claim 1, wherein X is N and Y is CH.

3. The compound of claim 1, wherein R¹ is H, C₁-C₄ alkyl, C₁-C₄ fluoroalkyl, or Y and R¹ are joined to form a C₂-C₃ alkylene, C₂-C₃ alkenylene, or thio-C₁-C₂ alkylene linkage between Y and the amine nitrogen to which R¹ is attached.

4. The compound of claim 1, wherein R¹ is H, methyl, ethyl, fluoromethyl, fluoroethyl, or Y and R¹ are joined to form a C₂-C₃ alkylene or thio-C₁-C₂ alkylene linkage between Y and the amine nitrogen to which R¹ is attached.

5. The compound of claim 1, wherein R¹ and Y are joined to form a C₂-C₃ alkylene, C₂-C₃ alkenylene, or thio-C₁-C₂ alkylene linkage between Y and the amine nitrogen to which R¹ is attached.

6. The compound of claim 1, wherein R² is H, OH, C₁-C₆ alkyl, C₁-C₆ alkoxy, or C₆-C₁₂ aryl.

7. The compound of claim 1, wherein R² is H, OH, C₁-C₆ alkoxy, or C₁-C₆ haloalkoxy.

8. The compound of claim 1, wherein R² is H, OCH₃, or OH.

9. The compound of claim 1, wherein
X is N and Y is CH;
R¹ is methyl; and
R² is OH, OCH₃ or H.

10. The compound of claim 1, wherein the compound is radiolabeled.

11. The compound of claim 10, wherein the radiolabel is ¹⁹F, ¹³C, ¹⁸F, ¹¹C, ⁷⁵Br, ⁷⁶Br, or ¹²³I.

12. The compound of claim 10, wherein the radiolabel is ¹⁸F or ¹¹C.

13. The compound of claim 10, wherein the F at the position adjacent to X in the phenyl ring in Formula 1 is ¹⁸F.

14. A pharmaceutical composition comprising the compound of claim 1 and a pharmaceutically acceptable carrier.

15. A method of detecting amyloid deposits in a patient, comprising administering to the patient a pharmaceutical composition comprising a detectable quantity of a compound of claim 1, and detecting the compound in the subject.

16. The method of claim 15, wherein detecting comprises imaging of the brain of the subject.

17. The method of claim 15, wherein the detectable quantity is an imaging effective quantity, and wherein detection is by a nuclear medicine imaging technique.

18. The method of claim 17, wherein the nuclear medicine imaging technique is magnetic resonance spectroscopy, magnetic resonance imaging, magnetic resonance imaging, positron emission tomography, or single-photon emission computed tomography.

19. The method of claim 15, wherein the patient is suspected of having dementia.

20. The method of claim 15, wherein the patient is being treated for dementia and the method further comprises determining the effectiveness of the treatment for reducing or preventing amyloid deposition.

21. A method of detecting and/or quantifying amyloid in biopsy or post-mortem tissue, comprising
contacting a preparation of biopsy or post-mortem tissue with a compound according to claim 1, wherein the compound comprises a detectable label, and
detecting the compound.

22. The method of claim 21, wherein the amount of bound compound of claim 1 is quantitated by comparison to a standard curve of compound incubated with known amounts of amyloid.

23. The method of claim 21, wherein detection is by a microscopic technique.
